(19) United States
(12) Patent Application Publication
(10) Pub. No.: US 2014/0307070 A1
(43) Pub. Date: Oct. 16, 2014

(54) SYSTEMS AND METHODS FOR SOUNDING A MESSAGE IDENTIFYING A CONTENT SOURCE TO A USER DURING AN ADVERTISEMENT

(71) Applicant: UNITED VIDEO PROPERTIES, INC., Santa Clara, CA (US)

(72) Inventor: Thomas Woods, Arlington Heights, IL (US)

(73) Assignee: United Video Properties, Inc., Santa Clara, CA (US)

(21) Appl. No.: 13/859,867

(22) Filed: Apr. 10, 2013

(51) Int. Cl. G09B 21/00; A61F 9/08

(52) U.S. Cl. CPC G09B 21/006; A61F 9/08
USPC 348/62

(57) ABSTRACT

Systems and methods for assisting a user in content selection are provided. A user request to access a content source may be received. Content from the content source may be received in response to the user request. A determination may be made as to whether the content received from the content source is an advertisement. An identifier of the content source may be retrieved from memory. The identifier of the content source may be cross-referenced with a database of audible messages to retrieve an audible message associated with the content source. The audible message may be played to identify the content source to the user in response to determining that the content received from the content source is an advertisement.

### BACKGROUND

Traditional media systems provide audio prompts that describe the program currently being presented on a given channel. These traditional systems also consider whether a program is audio friendly when providing the prompts. Such audio prompts can be very distracting and are not always helpful since visually-impaired users can infer what is on a channel based on the audio of the program received from the channel. However, when the program is in a commercial break, listening to just the audio of the program may confuse a visually-impaired user as to what program is scheduled to be shown on the channel.

### SUMMARY

These and other objects are accomplished in accordance with the principles of the present invention by sounding a message identifying a content source to a user during an advertisement.

In some embodiments, a user request to access a content source may be received. For example, a user may select a media asset listing from a plurality of media asset listings presented in a grid. The content source that provides the media asset corresponding to the selected listing may be identified and accessed. In some implementations, the content source may be a television channel. The user may select the television channel by selecting a program listing corresponding to a program provided by the television channel. In some implementations, the content source may be selected by the user pressing a channel change button or issuing a channel change command (e.g., a channel up/down command).

In some embodiments, content from the content source may be received in response to the user request. In some implementations, when the content source is a television channel, the tuner may be tuned to the corresponding television channel. The video and audio component of the program received from the television channel may be decoded and presented to the user. In some implementations, the content source may be an on-demand or Internet source, in which case suitable circuitry is instructed to access content from the selected source (e.g., a website address may be accessed using a network device).

In some embodiments, a determination may be made as to whether the content received from the content source includes an advertisement. In some implementations, a media asset may be scheduled for transmission from the content source during a time when the user request to access the content source is received. The media asset may include a commercial segment and a plot segment. In some implementations, the content received from the content source may be determined to include an advertisement when the current content being received is the commercial segment of the media asset.

In some implementations, information associated with the content may be processed to detect whether the content includes an advertisement. In some implementations, the information that is processed may include closed-caption information. In some embodiments, the advertisement may be determined to be included with the content in response to detecting the absence of closed-caption information. In some embodiments, the advertisement may be determined to be included with the content by comparing a current time with a scheduled transmission time of the content. The advertisement may be expected to be received at a given period within the scheduled transmission time of the content. When the current time matches the given period, the system may determine that the content being received includes an advertisement.

In some embodiments, an identifier of the content source may be retrieved from memory. In some embodiments, the identifier may be retrieved from a program listing associated with the content being accessed on the content source. In some embodiments, the identifier of the content source may be cross-referenced with a database of audible messages to retrieve an audible message associated with the content source. Each content source in the database may be associated with multiple audible messages. In some embodiments, a given one of the multiple audible messages may be selected for output to the user based on a user profile, a user selection, and/or a default setting. The audible messages may include personalized messages (e.g., a message having a user's name), content source name or numbers (e.g., ESPN or channel 2), an indication that the content being received includes an advertisement, and/or content source type (e.g., sports channel). The selected or identified audible message may be played to identify the content source to the user in response to determining that the content received from the content source is an advertisement. In some implementations, the audible message is sounded for the user before an audio portion of the content is output to the user. In some implementations, sounding of the audible message is prevented in response to determining that the content received from the content source is not an advertisement.

In some embodiments, a user input is received to enable an accessibility setting. The audible message may be played when the accessibility setting is enabled. The audible message is prevented from being played when the accessibility setting is disabled. The accessibility setting may be manually set by the user or may be automatically enabled by the system.

### BRIEF DESCRIPTION OF THE DRAWINGS

The above and other features of the present invention, its nature and various advantages will be more apparent upon consideration of the following detailed description, taken in conjunction with the accompanying drawings, and in which:

FIG. 1 is a diagram of an interactive grid display in accordance with some embodiments of the disclosure;

FIG. 2 is a diagram of an interactive media system in accordance with some embodiments of the disclosure;

FIG. 3 is a block diagram of an illustrative user equipment device in accordance with some embodiments of the disclosure;

FIG. 4 is a block diagram of an illustrative media system in accordance with some embodiments of the disclosure;

FIG. 5 is a flow diagram describing the process of sounding a message identifying a content source to a user during an advertisement in accordance with some embodiments of the disclosure; and

FIG. 6 is a diagram of an illustrative database of audible messages in accordance with some embodiments of the disclosure.

### DETAILED DESCRIPTION OF EMBODIMENTS

The amount of content available to users in any given content delivery system can be substantial. Consequently, many users desire a form of media guidance through an interface that allows users to efficiently navigate content selections and easily identify content that they may desire. An application that provides such guidance is referred to herein as an interactive media guidance application or, sometimes, a media guidance application or a guidance application.

Interactive media guidance applications may take various forms depending on the content for which they provide guidance. One typical type of media guidance application is an interactive television program guide. Interactive television program guides (sometimes referred to as electronic program guides) are well-known guidance applications that, among other things, allow users to navigate among and locate many types of content or media assets. Interactive media guidance applications may generate graphical user interface screens that enable a user to navigate among, locate and select content. As referred to herein, the terms “media asset” and “content” should be understood to mean an electronically consumable user asset, such as television programming, as well as pay-per-view programs, on-demand programs (as in video-on-demand (VOD) systems), Internet content (e.g., streaming content, downloadable content, Webcasts, etc.), video clips, audio, content information, pictures, rotating images, documents, playlists, websites, articles, books, electronic books, blogs, advertisements, chat sessions, social media, applications, games, and/or any other media or multimedia and/or combination of the same. Guidance applications also allow users to navigate among and locate content. As referred to herein, the term “multimedia” should be understood to mean content that utilizes at least two different content forms described above, for example, text, audio, images, video, or interactivity content forms. Content may be recorded, played, displayed or accessed by user equipment devices, but can also be part of a live performance.

Each media asset may include or be associated with certain subject matter. The subject matter or content included in or associated with a given media asset may change while the media asset is being presented. For example, one portion of the media asset may include commercials/advertisements (e.g., one form or type of subject matter) and another portion of the media asset may include portions of the plot. In another example, the media asset may include an action segment in one portion and a drama segment in another. Subject matter may be predefined in a media asset data structure received from a remote source (as discussed below). In some implementations, subject matter may be determined on-the-fly automatically based on an analysis of the content being presented to the user. For example, closed-caption information may be processed to determine the subject matter of the content being presented.

As referred to herein the phrase “subject matter” refers to any content criterion or characteristic associated with the media asset. For example, subject matter may indicate whether a portion of a media asset is a commercial/advertisement segment, whether a portion of a media asset is a plot segment, a type of the media asset, category of the media asset, genre of the media asset, content source of the media asset (e.g., broadcast source, Internet source, on-demand source, music type of source, music type of channel, video type of source, video type channel, etc.), time of day (e.g., morning, afternoon, evening), or any combination thereof.

With the advent of the Internet, mobile computing, and high-speed wireless networks, users are accessing media on user equipment devices on which they traditionally did not. As referred to herein, the phrase “user equipment device,” “user equipment,” “user device,” “electronic device,” “electronic equipment,” “media equipment device,” or “media device” should be understood to mean any device for accessing the content described above, such as a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a hand-held computer, a stationary telephone, a personal digital assistant (PDA), a mobile telephone, a portable video player, a portable music player, a portable gaming machine, a smart phone, or any other television equipment, computing equipment, or wireless device, and/or combination of the same. In some embodiments, the user equipment device may have a front facing screen and a rear facing screen, multiple front screens, or multiple angled screens. In some embodiments, the user equipment device may have a front facing camera and/or a rear facing camera. On these user equipment devices, users may be able to navigate among and locate the same content available through a television. Consequently, media guidance may be available on these devices, as well. The guidance provided may be for content available only through a television, for content available only through one or more of other types of user equipment devices, or for content available both through a television and one or more of the other types of user equipment devices. The media guidance applications may be provided as on-line applications (i.e., provided on a web-site), or as stand-alone applications or clients on user equipment devices. Various devices and platforms that may implement media guidance applications are described in more detail below.

One of the functions of the media guidance application is to provide media guidance data to users. As referred to herein, the phrase, “media guidance data” or “guidance data” should be understood to mean any data related to content, such as media listings, media-related information (e.g., broadcast times, broadcast channels, titles, descriptions, ratings information (e.g., parental control ratings, critic's ratings, etc.), genre or category information, actor information, logo data for broadcasters' or providers' logos, etc.), media format (e.g., standard definition, high definition, 3D, etc.), advertisement information (e.g., text, images, media clips, etc.), on-demand information, blogs, websites, subject matter information, and any other type of guidance data that is helpful for a user to navigate among and locate desired content selections.

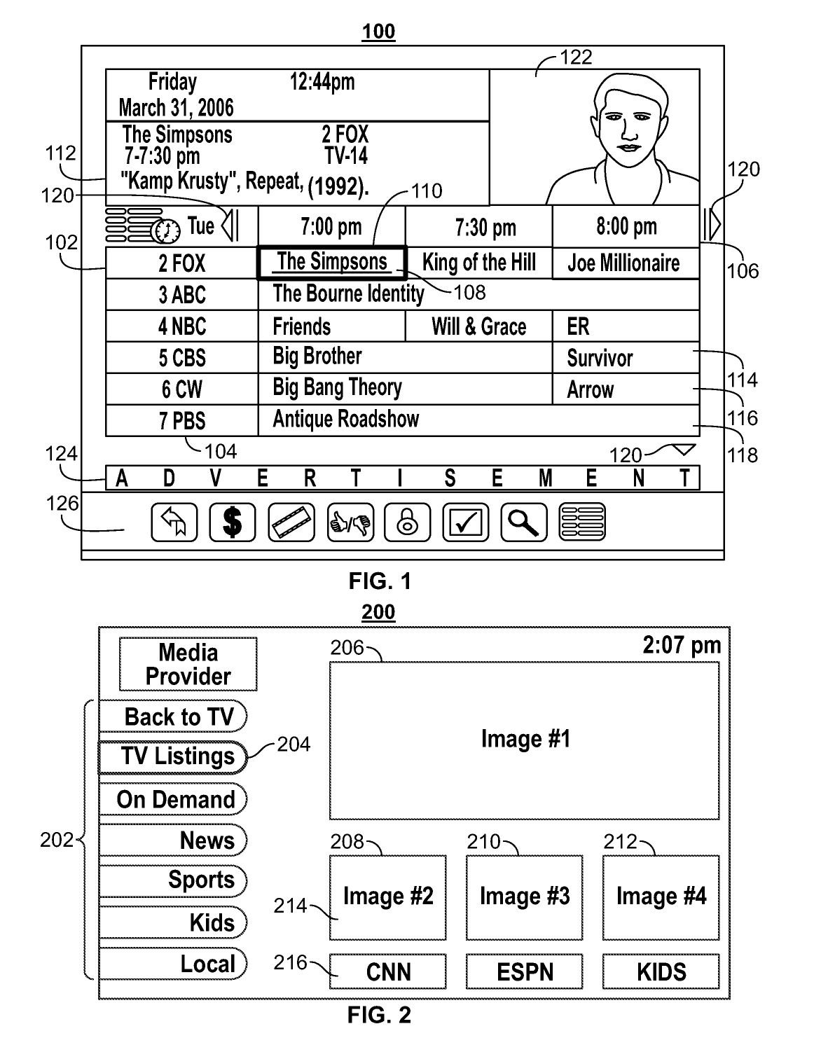

FIGS. 1-2 show illustrative display screens that may be used to provide media guidance data. The display screens shown in FIGS. 1-2 may be implemented on any suitable user equipment device or platform. While the displays of FIGS. 1-2 are illustrated as full screen displays, they may also be fully or partially overlaid over content being displayed. A user may indicate a desire to access content information by selecting a selectable option provided in a display screen (e.g., a menu option, a listings option, an icon, a hyperlink, etc.) or pressing a dedicated button (e.g., a GUIDE button) on a remote control or other user input interface or device. In response to the user's indication, the media guidance application may provide a display screen with media guidance data organized in one of several ways, such as by time and channel in a grid, by time, by channel, by source, by content type, by category (e.g., movies, sports, news, children, or other categories of programming), or other predefined, user-defined, or other organization criteria. The organization of the media guidance data is determined by guidance application data. As referred to herein, the phrase, “guidance application data” should be understood to mean data used in operating the guidance application, such as program information, guidance application settings, user preferences, or user profile information.

FIG. 1 shows illustrative grid program listings display 100 arranged by time and channel that also enables access to different types of content in a single display. Display 100 may include grid 102 with: (1) a column of channel/content type identifiers 104, where each channel/content type identifier (which is a cell in the column) identifies a different channel or content type available or media asset characteristics; and (2) a row of time identifiers 106, where each time identifier (which is a cell in the row) identifies a time block of programming. The media asset characteristics indicated in column of channel/content type identifiers 104 may include any combination of criteria that identifies a plurality of media assets (e.g., content source of a respective one of the media assets, a content type of the respective one of the media assets, or a content category of the respective one of the media assets). Although grid 102 is a by time-channel grid, any other type of grid may be provided such that time is shown on one dimension and a media characteristic is shown on another. For example, grid 102 may be arranged so that time is shown as in grid 102 and the column of identifiers may represent categories of different media assets or types of content sources (e.g., satellite and Internet). In particular, instead of showing the different programs available on a given channel (e.g., channel 2, FOX) at various times, grid 102 may display different programs that are of the genre or category (comedy) at various times regardless of what source they are received from.

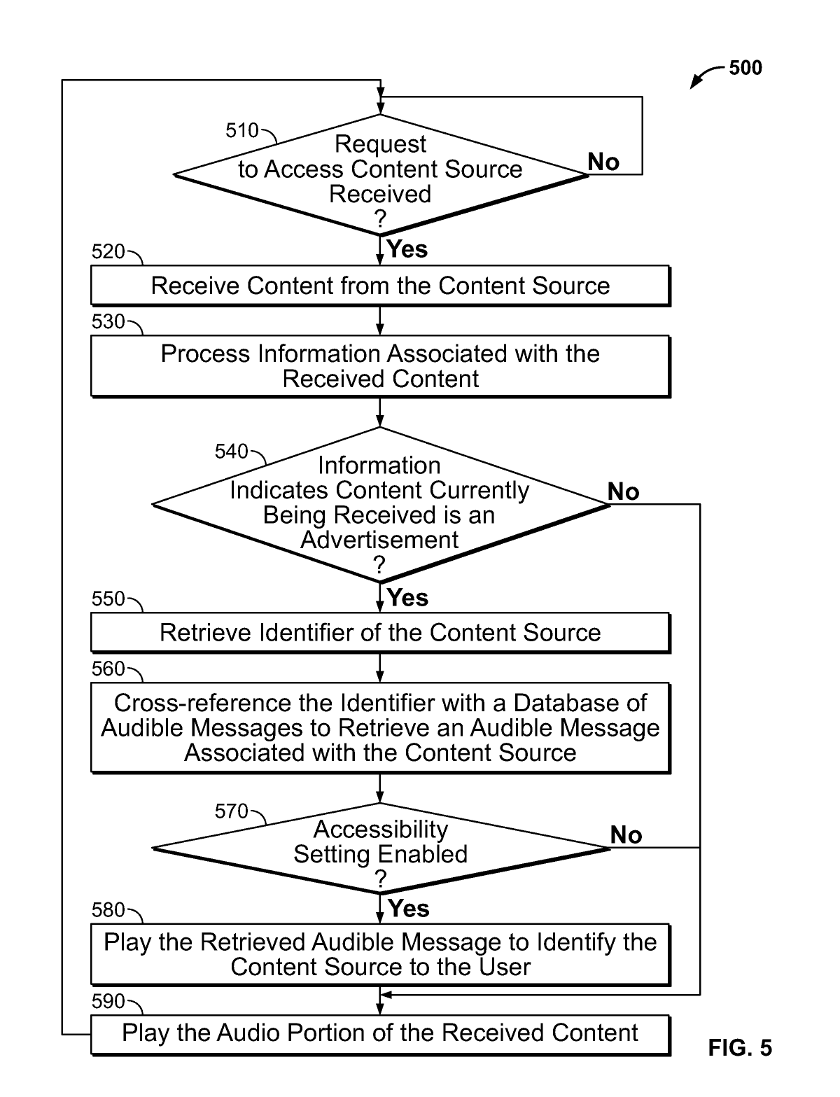

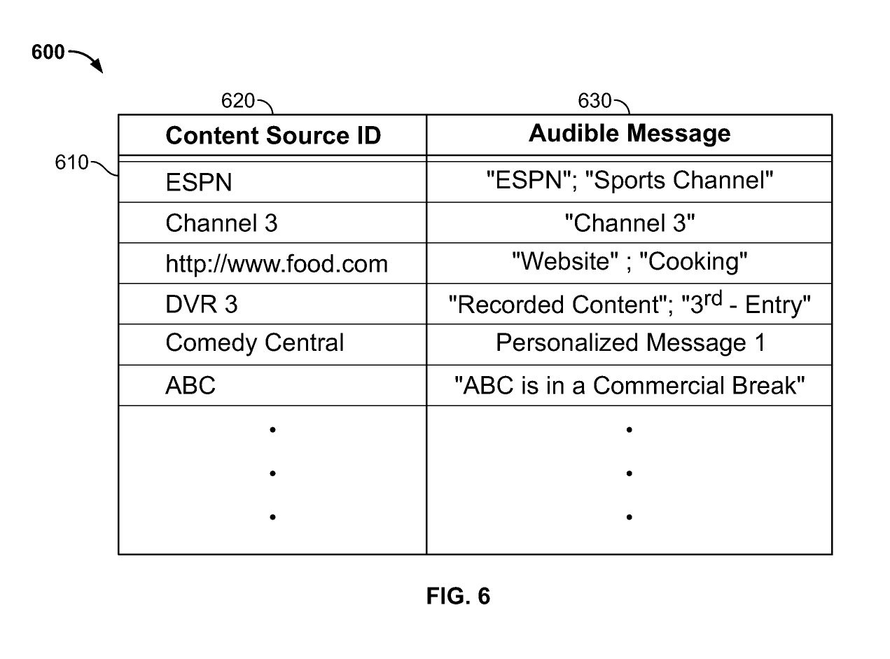

Grid 102 also includes cells of program listings, such as program listing 108, where each listing provides the title of the program provided on the listing's associated channel and time. As referred to herein, the term “program” should be understood to be synonymous with “media”. With a user input device, a user can select program listings by moving highlight region 110. Information relating to the program listing selected by highlight region 110 may be provided in program information region 112. Alternatively or in addition, as discussed below in connection with FIGS. 5 and 6, the information may be provided using other cells corresponding to the same transmission time as a program corresponding to the selected listing. Region 112 may include, for example, the program title, the program description, the time the program is provided (if applicable) (e.g., the transmission time of the program), the channel or content source of the program (if applicable), the program's rating, and other desired information.

In addition to providing access to linear programming (e.g., content that is scheduled to be transmitted to a plurality of user equipment devices at a predetermined transmission time and is provided according to a schedule), the media guidance application also provides access to non-linear programming (e.g., content accessible to a user equipment device at any time and is not provided according to a schedule). Non-linear programming may include content from different content sources including on-demand content (e.g., VOD), Internet content (e.g., streaming media, downloadable media, etc.), locally stored content (e.g., content stored on any user equipment device described above or other storage device), or other time-independent content. On-demand content may include movies or any other content provided by a particular content provider (e.g., HBO On Demand providing “The Sopranos” and “Curb Your Enthusiasm”). HBO ON DEMAND is a service mark owned by Time Warner Company L. P. et al. and THE SOPRANOS and CURB YOUR ENTHUSIASM are trademarks owned by the Home Box Office, Inc. Internet content may include web events, such as a chat session or Webcast, or content available on-demand as streaming content or downloadable content through an Internet web site or other Internet access (e.g., FTP).

Grid 102 may provide media guidance data for non-linear programming including on-demand listings, recorded content listings, and Internet content listings. A display combining media guidance data for content from different types of content sources is sometimes referred to as a “mixed-media” display. Various permutations of the types of media guidance data that may be displayed that are different than display 100 may be based on user selection or guidance application definition (e.g., a display of only recorded and broadcast listings, only on-demand and broadcast listings, etc.). In some embodiments, listings for these content types may be included directly in grid 102. Additional media guidance data may be displayed in response to the user selecting one of the navigational icons 120. (Pressing an arrow key on a user input device may affect the display in a similar manner as selecting navigational icons 120.) Specifically, the user may navigate along the time dimension to later or earlier times using icons 120.

Display 100 may also include video region 122, advertisement 124, and options region 126. Video region 122 may allow the user to view and/or preview programs that are currently available, will be available, or were available to the user. The content of video region 122 may correspond to, or be independent from, one of the listings displayed in grid 102. Grid displays including a video region are sometimes referred to as picture-in-guide (PIG) displays. PIG displays and their functionalities are described in greater detail in Satterfield et al. U.S. Pat. No. 6,564,378, issued May 13, 2003 and Yuen et al. U.S. Pat. No. 6,239,794, issued May 29, 2001, which are hereby incorporated by reference herein in their entireties. PIG displays may be included in other media guidance application display screens of the embodiments described herein.

Advertisement 124 may provide an advertisement for content that, depending on a viewer's access rights (e.g., for subscription programming), is currently available for viewing, will be available for viewing in the future, or may never become available for viewing, and may correspond to, or be unrelated to one or more of the content listings in grid 102. Advertisement 124 may also be for products or services related or unrelated to the content displayed in grid 102. Advertisement 124 may be selectable and provide further information about content, provide information about a product or a service, enable purchasing of content, a product, or a service, provide content relating to the advertisement, etc. Advertisement 124 may be targeted based on a user's profile/preferences, monitored user activity, the type of display provided, or on other suitable targeted advertisement bases.

While advertisement 124 is shown as rectangular or banner shaped, advertisements may be provided in any suitable size, shape, and location in a guidance application display. For example, advertisement 124 may be provided as a rectangular shape that is horizontally adjacent to grid 102. This is sometimes referred to as a panel advertisement. In addition, advertisements may be overlaid over content or a guidance application display or embedded within a display. Advertisements may also include text, images, rotating images, video clips, or other types of content described above. Advertisements may be stored in a user equipment device having a guidance application, in a database connected to the user equipment, in a remote location (including streaming media servers), or on other storage means, or a combination of these locations. Providing advertisements in a media guidance application is discussed in greater detail in, for example, Knudson et al., U.S. Patent Application Publication No. 2003/0110499, filed Jan. 17, 2003; Ward, III et al. U.S. Pat. No. 6,756,997, issued Jun. 29, 2004; and Schein et al. U.S. Pat. No. 6,388,714, issued May 14, 2002, which are hereby incorporated by reference herein in their entireties. It will be appreciated that advertisements may be included in other media guidance application display screens of the embodiments described herein.

Options region 126 may allow the user to access different types of content, media guidance application displays, and/or media guidance application features. Options region 126 may be part of display 100 (and other display screens described herein), or may be invoked by a user by selecting an on-screen option or pressing a dedicated or assignable button on a user input device. The selectable options within options region 126 may concern features related to program listings in grid 102 or may include options available from a main menu display. Features related to program listings may include searching for other air times or ways of receiving a program, recording a program, enabling series recording of a program, setting program and/or channel as a favorite, purchasing a program, or other features. Options available from a main menu display may include VOD options, parental control options, Internet options, volume control options, cloud-based options, device synchronization options, second screen device options, accessibility settings options, options to access various types of media guidance data displays, options to subscribe to a premium service, options to edit a user's profile, options to access a browse overlay, or other options.

In some embodiments, selection of the accessibility settings options navigates a user to a screen that includes a plurality of accessibility settings. From this screen, the user can enable/disable various settings. For example, one of the accessibility settings that are displayed may include a setting for sounding audible messages (notifications or prompts) that identify a content source to a user. This option may only sound the audible message (notification or prompt) when a user requests access to a content source on which an advertisement or commercial segment is currently being shown. Enabling this option may instruct control circuitry 304 to determine whether an advertisement is currently being received from a selected content source. In response to determining that an advertisement is currently being received, control circuitry 304 may retrieve an audible message from a database and play the audible message to the user to identify the content source to the user. Disabling this option may instruct control circuitry 304 to prevent playing of the audible message to the user even though an advertisement is currently being received from a selected content source. The audio from the commercial/advertisement segment or other content being received from the content source may be played or sounded instead of the audible message.

The media guidance application may be personalized based on a user's preferences. A personalized media guidance application allows a user to customize displays and features to create a personalized “experience” with the media guidance application. This personalized experience may be created by allowing a user to input these customizations and/or by the media guidance application monitoring user activity to determine various user preferences. Users may access their personalized guidance application by logging in or otherwise identifying themselves to the guidance application. Customization of the media guidance application may be made in accordance with a user profile. The customizations may include varying presentation schemes (e.g., color scheme of displays, font size of text, etc.), aspects of content listings displayed (e.g., only HDTV or only 3D programming, user-specified broadcast channels based on favorite channel selections, re-ordering the display of channels, recommended content, etc.), desired recording features (e.g., recording or series recordings for particular users, recording quality, etc.), parental control settings, automatic dynamic volume rate change control, customized presentation of Internet content (e.g., presentation of social media content, e-mail, electronically delivered articles, etc.) and other desired customizations.

The media guidance application may allow a user to provide user profile information or may automatically compile user profile information. The media guidance application may, for example, monitor the content the user accesses and/or other interactions the user may have with the guidance application. Additionally, the media guidance application may obtain all or part of other user profiles that are related to a particular user (e.g., from other web sites on the Internet the user accesses, such as www.allrovi.com, from other media guidance applications the user accesses, from other interactive applications the user accesses, from another user equipment device of the user, etc.), and/or obtain information about the user from other sources that the media guidance application may access. As a result, a user can be provided with a unified guidance application experience across the user's different user equipment devices. This type of user experience is described in greater detail below in connection with FIG. 4. Additional personalized media guidance application features are described in greater detail in Ellis et al., U.S. Patent Application Publication No. 2005/0251827, filed Jul. 11, 2005, Boyer et al., U.S. Pat. No. 7,165,098, issued Jan. 16, 2007, and Ellis et al., U.S. Patent Application Publication No. 2002/0174430, filed Feb. 21, 2002, which are hereby incorporated by reference herein in their entireties.

Another display arrangement for providing media guidance is shown in FIG. 2. Video mosaic display 200 includes selectable options 202 for content information organized based on content type, genre, and/or other organization criteria. In display 200, television listings option 204 is selected, thus providing listings 206, 208, 210, and 212 as broadcast program listings. In display 200, the listings may provide graphical images including cover art, still images from the content, video clip previews, live video from the content, or other types of content that indicate to a user the content being described by the media guidance data in the listing. Each of the graphical listings may also be accompanied by text to provide further information about the content associated with the listing. For example, listing 208 may include more than one portion, including media portion 214 and text portion 216. Media portion 214 and/or text portion 216 may be selectable to view content in full-screen or to view information related to the content displayed in media portion 214 (e.g., to view listings for the channel that the video is displayed on).

The listings in display 200 are of different sizes (i.e., listing 206 is larger than listings 208, 210, and 212), but if desired, all the listings may be the same size. Listings may be of different sizes or graphically accentuated to indicate degrees of interest to the user or to emphasize certain content, as desired by the content provider or based on user preferences. Various systems and methods for graphically accentuating content listings are discussed in, for example, Yates, U.S. Patent Application Publication No. 2010/0153885, filed Dec. 29, 2005, which is hereby incorporated by reference herein in its entirety.

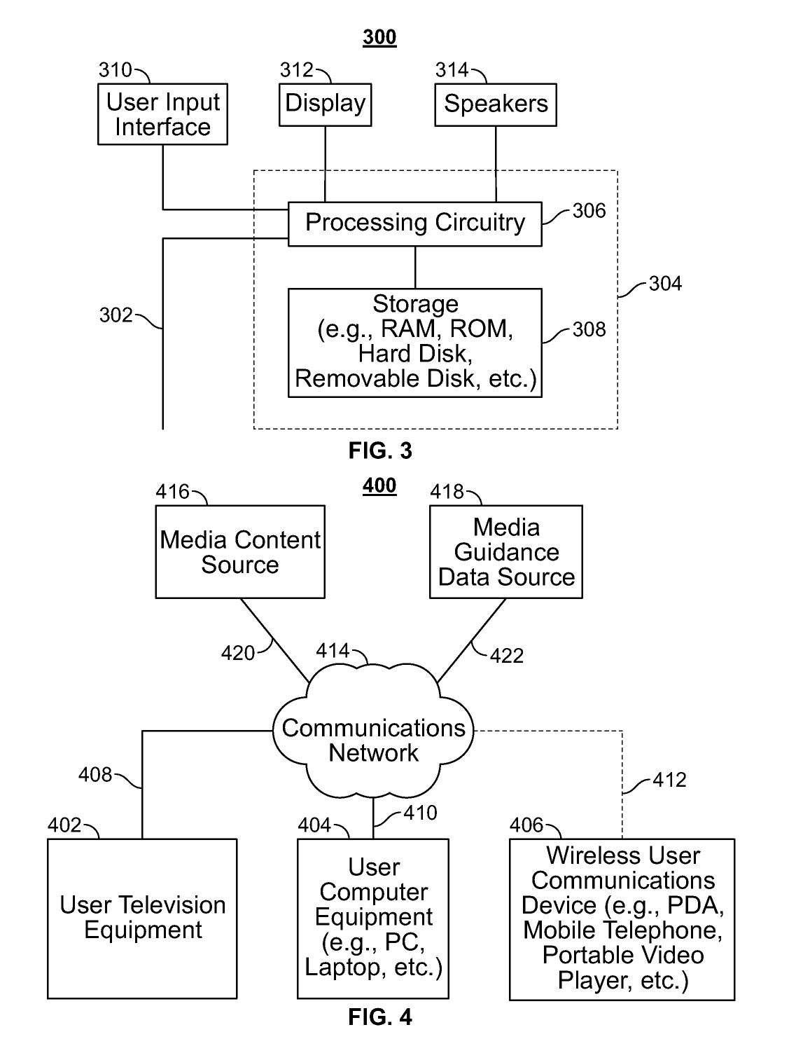

Users may access content and the media guidance application (and its display screens described above and below) from one or more of their user equipment devices. FIG. 3 shows a generalized embodiment of illustrative user equipment device 300. More specific implementations of user equipment devices are discussed below in connection with FIG. 4. User equipment device 300 may receive content and data via input/output (hereinafter “I/O”) path 302. I/O path 302 may provide content (e.g., broadcast programming, on-demand programming, Internet content, content available over a local area network (LAN) or wide area network (WAN), and/or other content) and data to control circuitry 304, which includes processing circuitry 306 and storage 308. Control circuitry 304 may be used to send and receive commands, requests, and other suitable data using I/O path 302. I/O path 302 may connect control circuitry 304 (and specifically processing circuitry 306) to one or more communications paths (described below). I/O functions may be provided by one or more of these communications paths, but are shown as a single path in FIG. 3 to avoid overcomplicating the drawing.

Control circuitry 304 may be based on any suitable processing circuitry such as processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiples of the same type of processing units (e.g., two Intel Core i7 processors) or multiple different processors (e.g., an Intel Core i5 processor and an Intel Core i7 processor). In some embodiments, control circuitry 304 executes instructions for a media guidance application stored in memory (i.e., storage 308). Specifically, control circuitry 304 may be instructed by the media guidance application to perform the functions discussed above and below. For example, the media guidance application may provide instructions to control circuitry 304 to generate the media guidance displays. In some implementations, any action performed by control circuitry 304 may be based on instructions received from the media guidance application.

In client-server based embodiments, control circuitry 304 may include communications circuitry suitable for communicating with a guidance application server or other networks or servers. The instructions for carrying out the above-mentioned functionality may be stored on the guidance application server. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths (which is described in more detail in connection with FIG. 4). In addition, communications circuitry may include circuitry that enables peer-to-peer communication of user equipment devices, or communication of user equipment devices in locations remote from each other (described in more detail below).

Memory may be an electronic storage device provided as storage 308 that is part of control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 308 may be used to store various types of content described herein as well as media guidance information, described above, and guidance application data, described above. Nonvolatile memory may also be used (e.g., to launch a boot-up routine and other instructions). Cloud-based storage, described in relation to FIG. 4, may be used to supplement storage 308 or instead of storage 308.

In some embodiments, a database of audible messages may be stored in storage 308. The database may include a mapping between a content source identifier (e.g., content source name, channel name or number) and a corresponding audible message (notification or prompt). The audible message (notification or prompt) may include a digitally encoded sound file (e.g., MP3, WAV, etc.) that when played back (or decoded) reads out the content source name, channel name or number, an indication that the content being received includes an advertisement, and/or a brief description of the type of content source or channel. For example, the audible message may provide instructions to control circuitry 304 to read out the channel name ESPN when the channel being accessed is ESPN. Similarly, the audible message may provide instructions to control circuitry 304 to read out the content source name NETFLIX or Internet when the content source being accessed is an Internet content source or other on-demand video delivery content source. As referred to herein, the phrase “audible message” is used interchangeably with “audible notification” and “audible prompt” and should be understood to have the same meaning.

Control circuitry 304 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (e.g., for converting over-the-air, analog, or digital signals to MPEG signals for storage) may also be provided. Control circuitry 304 may also include scaler circuitry for upconverting and downconverting content into the preferred output format of the user equipment 300. Circuitry 304 may also include digital-to-analog converter circuitry and analog-to-digital converter circuitry for converting between digital and analog signals. The tuning and encoding circuitry may be used by the user equipment device to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (e.g., watch and record functions, picture-in-picture (PIP) functions, multiple-tuner recording, etc.). If storage 308 is provided as a separate device from user equipment 300, the tuning and encoding circuitry (including multiple tuners) may be associated with storage 308.

A user may send instructions to control circuitry 304 using user input interface 310. User input interface 310 may be any suitable user interface, such as a remote control, mouse, trackball, keypad, keyboard, touch screen, touchpad, stylus input, joystick, voice recognition interface, or other user input interfaces. Display 312 may be provided as a stand-alone device or integrated with other elements of user equipment device 300. Display 312 may be one or more of a monitor, a television, a liquid crystal display (LCD) for a mobile device, or any other suitable equipment for displaying visual images. In some embodiments, display 312 may be HDTV-capable. In some embodiments, display 312 may be a 3D display, and the interactive media guidance application and any suitable content may be displayed in 3D. A video card or graphics card may generate the output to the display 312. The video card may offer various functions such as accelerated rendering of 3D scenes and 2D graphics, MPEG-2/MPEG-4 decoding, TV output, or the ability to connect multiple monitors. The video card may be any processing circuitry described above in relation to control circuitry 304. The video card may be integrated with the control circuitry 304. Speakers 314 may be provided as integrated with other elements of user equipment device 300 or may be stand-alone units. The audio component of videos and other content displayed on display 312 may be played through speakers 314. In some embodiments, the audio may be distributed to a receiver (not shown), which processes and outputs the audio via speakers 314. For example, speakers 314 may be used to sound an audible message that identifies a content source to the user and/or indicates that the content being received includes an advertisement.

The guidance application may be implemented using any suitable architecture. For example, it may be a stand-alone application wholly implemented on user equipment device 300. In such an approach, instructions of the application are stored locally, and data for use by the application is downloaded on a periodic basis (e.g., from an out-of-band feed, from an Internet resource, or using another suitable approach). In some embodiments, the media guidance application is a client-server based application. Data for use by a thick or thin client implemented on user equipment device 300 is retrieved on-demand by issuing requests to a server remote to the user equipment device 300. In one example of a client-server based guidance application, control circuitry 304 runs a web browser that interprets web pages provided by a remote server.

In some embodiments, the media guidance application is downloaded and interpreted or otherwise run by an interpreter or virtual machine (run by control circuitry 304). In some embodiments, the guidance application may be encoded in the ETV Binary Interchange Format (EBIF), received by control circuitry 304 as part of a suitable feed, and interpreted by a user agent running on control circuitry 304. For example, the guidance application may be an EBIF application. In some embodiments, the guidance application may be defined by a series of JAVA-based files that are received and run by a local virtual machine or other suitable middleware executed by control circuitry 304. In some of such embodiments (e.g., those employing MPEG-2 or other digital media encoding schemes), the guidance application may be, for example, encoded and transmitted in an MPEG-2 object carousel with the MPEG audio and video packets of a program.

User equipment device 300 of FIG. 3 can be implemented in system 400 of FIG. 4 as user television equipment 402, user computer equipment 404, wireless user communications device 406, or any other type of user equipment suitable for accessing content, such as a non-portable gaming machine. For simplicity, these devices may be referred to herein collectively as user equipment or user equipment devices, and may be substantially similar to user equipment devices described above. User equipment devices, on which a media guidance application may be implemented, may function as a standalone device or may be part of a network of devices. Various network configurations of devices may be implemented and are discussed in more detail below.

A user equipment device utilizing at least some of the system features described above in connection with FIG. 3 may not be classified solely as user television equipment 402, user computer equipment 404, or a wireless user communications device 406. For example, user television equipment 402 may, like some user computer equipment 404, be Internet-enabled allowing for access to Internet content, while user computer equipment 404 may, like some television equipment 402, include a tuner allowing for access to television programming. The media guidance application may have the same layout on various different types of user equipment or may be tailored to the display capabilities of the user equipment. For example, on user computer equipment 404, the guidance application may be provided as a website accessed by a web browser. In another example, the guidance application may be scaled down for wireless user communications devices 406.

In system 400, there is typically more than one of each type of user equipment device but only one of each is shown in FIG. 4 to avoid overcomplicating the drawing. In addition, each user may utilize more than one type of user equipment device and also more than one of each type of user equipment device.

In some embodiments, a user equipment device (e.g., user television equipment 402, user computer equipment 404, wireless user communications device 406) may be referred to as a “second screen device.” For example, a second screen device may supplement content presented on a first user equipment device. The content presented on the second screen device may be any suitable content that supplements the content presented on the first device. In some embodiments, the second screen device provides an interface for adjusting settings and display preferences of the first device. In some embodiments, the second screen device is configured for interacting with other second screen devices or for interacting with a social network. The second screen device can be located in the same room as the first device, a different room from the first device but in the same house or building, or in a different building from the first device.

The user may also set various settings to maintain consistent media guidance application settings across in-home devices and remote devices. Settings include those described herein, as well as channel and program favorites, programming preferences that the guidance application utilizes to make programming recommendations, display preferences, target volume levels, volume thresholds, volume adjustment rates, and other desirable guidance settings. For example, if a user sets a channel as a favorite on, for example, the website www.allrovi.com on their personal computer at their office, the same channel would appear as a favorite on the user's in-home devices (e.g., user television equipment and user computer equipment) as well as the user's mobile devices, if desired. Therefore, changes made on one user equipment device can change the guidance experience on another user equipment device, regardless of whether they are the same or a different type of user equipment device. In addition, the changes made may be based on settings input by a user, as well as user activity monitored by the guidance application.

The user equipment devices may be coupled to communications network 414. Namely, user television equipment 402, user computer equipment 404, and wireless user communications device 406 are coupled to communications network 414 via communications paths 408, 410, and 412, respectively. Communications network 414 may be one or more networks including the Internet, a mobile phone network, mobile voice or data network (e.g., a 4G or LTE network), cable network, public switched telephone network, or other types of communications network or combinations of communications networks. Paths 408, 410, and 412 may separately or together include one or more communications paths, such as, a satellite path, a fiber-optic path, a cable path, a path that supports Internet communications (e.g., IPTV), free-space connections (e.g., for broadcast or other wireless signals), or any other suitable wired or wireless communications path or combination of such paths. Path 412 is drawn with dotted lines to indicate that in the exemplary embodiment shown in FIG. 4 it is a wireless path and paths 408 and 410 are drawn as solid lines to indicate they are wired paths (although these paths may be wireless paths, if desired). Communications with the user equipment devices may be provided by one or more of these communications paths, but are shown as a single path in FIG. 4 to avoid overcomplicating the drawing.

Although communications paths are not drawn between user equipment devices, these devices may communicate directly with each other via communication paths, such as those described above in connection with paths 408, 410, and 412, as well as other short-range point-to-point communication paths, such as USB cables, IEEE 1394 cables, wireless paths (e.g., Bluetooth, infrared, IEEE 802-11x, etc.), or other short-range communication via wired or wireless paths. BLUETOOTH is a certification mark owned by Bluetooth SIG, INC. The user equipment devices may also communicate with each other directly through an indirect path via communications network 414.

System 400 includes content source 416 and media guidance data source 418 coupled to communications network 414 via communication paths 420 and 422, respectively. Paths 420 and 422 may include any of the communication paths described above in connection with paths 408, 410, and 412. Communications with the content source 416 and media guidance data source 418 may be exchanged over one or more communications paths, but are shown as a single path in FIG. 4 to avoid overcomplicating the drawing. In addition, there may be more than one of each of content source 416 and media guidance data source 418, but only one of each is shown in FIG. 4 to avoid overcomplicating the drawing. (The different types of each of these sources are discussed below.) If desired, content source 416 and media guidance data source 418 may be integrated as one source device. Although communications between sources 416 and 418 with user equipment devices 402, 404, and 406 are shown as through communications network 414, in some embodiments, sources 416 and 418 may communicate directly with user equipment devices 402, 404, and 406 via communication paths (not shown) such as those described above in connection with paths 408, 410, and 412.

Content source 416 may include one or more types of content distribution equipment including a television distribution facility, cable system headend, satellite distribution facility, programming sources (e.g., television broadcasters, such as NBC, ABC, HBO, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Content source 416 may be the originator of content (e.g., a television broadcaster, a Webcast provider, etc.) or may not be the originator of content (e.g., an on-demand content provider, an Internet provider of content of broadcast programs for downloading, etc.). Content source 416 may include cable sources, satellite providers, on-demand providers, Internet providers, over-the-top content providers, or other providers of content. Content source 416 may also include a remote media server used to store different types of content (including video content selected by a user), in a location remote from any of the user equipment devices. Systems and methods for remote storage of content, and providing remotely stored content to user equipment are discussed in greater detail in connection with Ellis et al., U.S. Pat. No. 7,761,892, issued Jul. 20, 2010, which is hereby incorporated by reference herein in its entirety.

Media guidance data source 418 may provide media guidance data, such as the media guidance data described above. Media guidance application data may be provided to the user equipment devices using any suitable approach. In some embodiments, the guidance application may be a stand-alone interactive television program guide that receives program guide data via a data feed (e.g., a continuous feed or trickle feed). Program schedule data and other guidance data may be provided to the user equipment on a television channel sideband, using an in-band digital signal, using an out-of-band digital signal, or by any other suitable data transmission technique. Program schedule data and other media guidance data may be provided to user equipment on multiple analog or digital television channels. In some embodiments, a database that maps content source identifiers to audible messages that are stored in storage 308 may be received from data source 418.

In some embodiments, guidance data from media guidance data source 418 may be provided to users' equipment using a client-server approach. For example, a user equipment device may pull media guidance data from a server, or a server may push media guidance data to a user equipment device. In some embodiments, a guidance application client residing on the user's equipment may initiate sessions with source 418 to obtain guidance data when needed, e.g., when the guidance data is out of date or when the user equipment device receives a request from the user to receive data. Media guidance may be provided to the user equipment with any suitable frequency (e.g., continuously, daily, a user-specified period of time, a system-specified period of time, in response to a request from user equipment, etc.). Media guidance data source 418 may provide user equipment devices 402, 404, and 406 the media guidance application itself or software updates for the media guidance application.

Media guidance applications may be, for example, stand-alone applications implemented on user equipment devices. For example, the media guidance application may be implemented as software or a set of executable instructions which may be stored in storage 308, and executed by control circuitry 304 of a user equipment device 300. In some embodiments, media guidance applications may be client-server applications where only a client application resides on the user equipment device, and server application resides on a remote server. For example, media guidance applications may be implemented partially as a client application on control circuitry 304 of user equipment device 300 and partially on a remote server as a server application (e.g., media guidance data source 418) running on control circuitry of the remote server. When executed by control circuitry of the remote server (such as media guidance data source 418), the media guidance application may instruct control circuitry 304 to generate the guidance application displays and transmit the generated displays to the user equipment devices. The server application may instruct control circuitry 304 of the media guidance data source 418 to transmit data for storage on the user equipment. The client application may instruct control circuitry 304 of the receiving user equipment to generate the guidance application displays.

Content and/or media guidance data delivered to user equipment devices 402, 404, and 406 may be over-the-top (OTT) content. OTT content delivery allows Internet-enabled user devices, including any user equipment device described above, to receive content that is transferred over the Internet, including any content described above, in addition to content received over cable or satellite connections. OTT content is delivered via an Internet connection provided by an Internet service provider (ISP), but a third party distributes the content. The ISP may not be responsible for the viewing abilities, copyrights, or redistribution of the content, and may only transfer IP packets provided by the OTT content provider. Examples of OTT content providers include YOUTUBE, NETFLIX, and HULU, which provide audio and video via IP packets. YouTube is a trademark owned by Google Inc., Netflix is a trademark owned by Netflix Inc., and Hulu is a trademark owned by Hulu, LLC. OTT content providers may additionally or alternatively provide media guidance data described above. In addition to content and/or media guidance data, providers of OTT content can distribute media guidance applications (e.g., web-based applications or cloud-based applications), or the content can be displayed by media guidance applications stored on the user equipment device.

Media guidance system 400 is intended to illustrate a number of approaches, or network configurations, by which user equipment devices and sources of content and guidance data may communicate with each other for the purpose of accessing content and providing media guidance. The embodiments described herein may be applied in any one or a subset of these approaches, or in a system employing other approaches for delivering content and providing media guidance. The following four approaches provide specific illustrations of the generalized example of FIG. 4.

In one approach, user equipment devices may communicate with each other within a home network. User equipment devices can communicate with each other directly via short-range point-to-point communication schemes described above, via indirect paths through a hub or other similar device provided on a home network, or via communications network 414. Each of the multiple individuals in a single home may operate different user equipment devices on the home network. As a result, it may be desirable for various media guidance information or settings to be communicated between the different user equipment devices. For example, it may be desirable for users to maintain consistent media guidance application settings on different user equipment devices within a home network, as described in greater detail in Ellis et al., U.S. patent application Ser. No. 11/179,410, filed Jul. 11, 2005. Different types of user equipment devices in a home network may also communicate with each other to transmit content. For example, a user may transmit content from user computer equipment to a portable video player or portable music player.

In a second approach, users may have multiple types of user equipment by which they access content and obtain media guidance. For example, some users may have home networks that are accessed by in-home and mobile devices. Users may control in-home devices via a media guidance application implemented on a remote device. For example, users may access an online media guidance application on a website via a personal computer at their office, or a mobile device such as a PDA or web-enabled mobile telephone. The user may set various settings (e.g., recordings, reminders, or other settings) on the online guidance application to control the user's in-home equipment. The online guide may control the user's equipment directly, or by communicating with a media guidance application on the user's in-home equipment. Various systems and methods for user equipment devices communicating, where the user equipment devices are in locations remote from each other, is discussed in, for example, Ellis et al., U.S. Pat. No. 8,046,801, issued Oct. 25, 2011, which is hereby incorporated by reference herein in its entirety.

In a third approach, users of user equipment devices inside and outside a home can use their media guidance application to communicate directly with content source 416 to access content. Specifically, within a home, users of user television equipment 402 and user computer equipment 404 may access the media guidance application to navigate among and locate desirable content. Users may also access the media guidance application outside of the home using wireless user communications devices 406 to navigate among and locate desirable content.

In a fourth approach, user equipment devices may operate in a cloud computing environment to access cloud services. In a cloud computing environment, various types of computing services for content sharing, storage or distribution (e.g., video sharing sites or social networking sites) are provided by a collection of network-accessible computing and storage resources, referred to as “the cloud.” For example, the cloud can include a collection of server computing devices, which may be located centrally or at distributed locations, which provide cloud-based services to various types of users and devices connected via a network such as the Internet via communications network 414. These cloud resources may include one or more content sources 416 and one or more media guidance data sources 418. In addition or in the alternative, the remote computing sites may include other user equipment devices, such as user television equipment 402, user computer equipment 404, and wireless user communications device 406. For example, the other user equipment devices may provide access to a stored copy of a video or a streamed video. In such embodiments, user equipment devices may operate in a peer-to-peer manner without communicating with a central server.

The cloud provides access to services, such as content storage, content sharing, or social networking services, among other examples, as well as access to any content described above, for user equipment devices. Services can be provided in the cloud through cloud computing service providers, or through other providers of online services. For example, the cloud-based services can include a content storage service, a content sharing site, a social networking site, or other services via which user-sourced content is distributed for viewing by others on connected devices. These cloud-based services may allow a user equipment device to store content to the cloud and to receive content from the cloud rather than storing content locally and accessing locally-stored content.

A user may use various content capture devices, such as camcorders, digital cameras with video mode, audio recorders, mobile phones, and handheld computing devices, to record content. The user can upload content to a content storage service on the cloud either directly, for example, from user computer equipment 404 or wireless user communications device 406 having content capture feature. Alternatively, the user can first transfer the content to a user equipment device, such as user computer equipment 404. The user equipment device storing the content uploads the content to the cloud using a data transmission service on communications network 414. In some embodiments, the user equipment device itself is a cloud resource, and other user equipment devices can access the content directly from the user equipment device on which the user stored the content.

Cloud resources may be accessed by a user equipment device using, for example, a web browser, a media guidance application, a desktop application, a mobile application, and/or any combination of access applications of the same type. The user equipment device may be a cloud client that relies on cloud computing for application delivery, or the user equipment device may have some functionality without access to cloud resources. For example, some applications running on the user equipment device may be cloud applications, i.e., applications delivered as a service over the Internet, while other applications may be stored and run on the user equipment device. In some embodiments, a user device may receive content from multiple cloud resources simultaneously. For example, a user device can stream audio from one cloud resource while downloading content from a second cloud resource. Or a user device can download content from multiple cloud resources for more efficient downloading. In some embodiments, user equipment devices can use cloud resources for processing operations such as the processing operations performed by processing circuitry described in relation to FIG. 3.

In some embodiments, a visually impaired user may browse content using user equipment device 300. The user may browse content by changing channels or selecting different content sources from grid 102 (FIG. 1). In response to receiving the user request to access a given content source (e.g., a channel change request or channel selection request), control circuitry 304 may determine whether the content received from the selected content source is an advertisement or a media asset in a commercial segment. In some embodiments, this determination may be performed only when an accessibility setting is enabled. Systems and methods for determining whether content is an advertisement or commercial are described in greater detail in Quan et al. U.S. Pat. No. 7,298,962, issued Nov. 20, 2007, Knee et al. U.S. Patent Application Publication No. 2010/0166389, and Craner et al. U.S. Patent Application Publication No. 2010/0119206, which are hereby incorporated by reference herein in their entireties. When control circuitry 304 determines that the content being received from the selected content source is an advertisement or a media asset in a commercial segment, control circuitry 304 may play an audible message for the user that identifies the content source and/or provides an indication that the content being received includes an advertisement. This audible message may be played before or instead of playing audio of the advertisement or the commercial segment of the media asset.

By playing an audible message instead of audio from an advertisement in response to selection of a content source, the system prevents confusing the visually-impaired user as to the audio being heard not being what is expected and lets the user know that the content source selected is in an advertisement break. The user may then choose to wait for the advertisement to end in order to wait for the media asset scheduled for transmission from the content source to start.

A visually impaired user may be able to determine what content source was selected just by listening to the content being provided. For example, when a content source that typically provides weather information (e.g., a weather channel) is selected, a visually impaired user can hear weather reports and is able to determine that the content source selected is of the type that provides weather reports. Similarly, when a content source that typically provides sports information (e.g., a sports channel) is selected, a visually impaired user can hear sports-related information and is able to determine that the content source selected is of the type that provides sports information. However, when such a content source is in a commercial break or is playing an advertisement, the visually-impaired user is unable to match the audio being heard to any type of expected content source. This may confuse the user, as the user is hearing information that is unexpected. Accordingly, control circuitry 304 in such circumstances first informs the user about what content source was selected and then provides the audio from the content source.

FIG. 5 is a flow diagram describing the process 500 of sounding a message identifying a content source to a user during an advertisement in accordance with some embodiments of the disclosure. At step 510, a determination is made as to whether a request to access a content source is received. In response to determining that a request to access a content source was received, the process proceeds to step 520, otherwise, the process proceeds to step 510.

In some embodiments, control circuitry 304 may receive a channel change request from the user as the request to access a content source. For example, the user may issue a channel up/down instruction to control circuitry 304 using user input interface 310. In response to receiving the channel up/down instruction, control circuitry 304 may instruct a tuner to tune to the next/previous channel. At step 520, control circuitry 304 may receive the content from the newly tuned channel and present the video and play the audio received from the channel for the user using device 300.

In some embodiments, control circuitry 304 may receive a channel selection from the user from grid 102 as the request to access a content source. For example, the user may highlight a given program listing and press a SELECT key using user input interface 310 which instructs control circuitry 304 to access the content source (e.g., channel, Internet source, website, or any other type of content source discussed above) corresponding to the given program listing. In response to receiving the selection of the program listing, control circuitry 304 may instruct a tuner to tune to the channel corresponding to the selected program listing and/or may instruct a communications circuit (e.g., network device) to access the content source (e.g., website, Internet source, or other type of source that is different from a channel discussed above) corresponding to the selected listing. At step 520, control circuitry 304 may receive the content from the selected content source and present the video and play the audio received from the selected content source for the user using device 300.

At step 530, information associated with the received content may be processed. In some embodiments, control circuitry 304 may process closed-caption information, an expected volume level, a data structure associated with the content being received, metadata associated with the received content, and/or any other data that can be processed to determine the presence or absence of a commercial or advertisement segment.

In some embodiments, control circuitry 304 may determine a title or content identifier of the content being received from the content source. Control circuitry 304 may retrieve from storage 308 a program listing for the content being presented by cross-referencing a content source identifier and current time with a database of schedule information. Using the determined title or content identifier, control circuitry 304 may retrieve a data structure from storage 308 corresponding to the title or content identifier. In some embodiments, control circuitry 304 may generate an SQL query that includes the current time and the content source being accessed. Control circuitry 304 may transmit the SQL query to a local or remote database. The local or remote database may identify a data structure associated with content provided on the content source at the specified time and may return the data structure back to control circuitry 304. The data structure may include multiple fields. For example, the data structure may include a title field that identifies the title of the accessed content, a related content field identifying related content, a scheduled transmission time field identifying the period of time during which the content is available from the content source, a detailed description field, fields that indicate whether the content being received includes advertisement breaks, fields that specify the times at which the advertisement breaks are received, and subject matter fields which specify different attributes associated with the content. Control circuitry 304 may retrieve the data structure for the currently presented content before, during, or after accessing the content source from which the content is being received and presented.

At step 540, control circuitry 304 may determine whether the processed information indicates content currently being received is an advertisement. In response to determining that the processed information indicates content currently being received is an advertisement, the process proceeds to step 550, otherwise, the process proceeds to step 590. In some embodiments, control circuitry 304 may determine using the retrieved data structure whether the content currently being received is an advertisement. In particular, control circuitry 304 may compare the current time to the field in the data structure that indicates when advertisement breaks are provided. When the current time falls within the period, control circuitry 304 may determine that the content currently being received is an advertisement.

In some embodiments, control circuitry 304 may analyze closed-caption data for the content being received from the selected content source to process the information associated with the received content. For example, control circuitry 304 may process text in currently received closed-caption data to determine the subject matter of the content being received. Specifically, control circuitry 304 may process text in currently received closed-caption data to determine whether the content being received is an advertisement at step 540. In some implementations, control circuitry 304 may determine whether the content being received is an advertisement based on the absence of closed-caption data. Specifically, when no closed-caption data is received with the content from the content source, control circuitry 304 may determine that the content currently being received is a commercial or advertisement segment. Whereas, when closed-caption data is presented or is being received, control circuitry 304 may determine that the content being received is a plot segment or is not an advertisement.

In some embodiments, control circuitry 304 may analyze the subject matter of the media asset scheduled for transmission from the selected content source to process the information associated with the received content. For example, control circuitry 304 may retrieve the subject matter of the media asset scheduled for transmission from a data structure field associated with the media asset. Based on the subject matter of the media asset, control circuitry 304 may retrieve an expected time period for advertisements or commercial breaks for the type of subject matter. For example, a news type of media asset may have advertisement breaks every 10 minutes following the start of the media asset. Control circuitry 304 may determine what time the media asset started and compare that time with the current time. In response to determining that the current time falls within the expected time period for advertisements for the type of media asset scheduled for transmission, control circuitry 304 may determine that the content currently being received is a commercial or advertisement segment.

In some embodiments, control circuitry 304 may analyze the volume level of the content being received from the selected content source to process the information associated with the received content. For example, control circuitry 304 may retrieve the currently set volume level and compare that volume level to the volume level being output. When the volume level being output is greater than the expected volume level for the currently set volume level, control circuitry 304 may determine that the content currently being received is a commercial or advertisement segment.

At step 550, control circuitry 304 may retrieve an identifier of the content source. For example, control circuitry 304 may process information stored in a program listing for the content source to identify a name or number that identifies the content source (e.g., channel number).

At step 560, control circuitry 304 may cross-reference the content source identifier with a database of audible messages to retrieve an audible message associated with the content source. In some embodiments, control circuitry 304 may generate an SQL query that includes the identifier of the content source. Control circuitry 304 may transmit the SQL query to a local or remote database. The local or remote database may process the entries (shown in FIG. 6), identify one or more audible messages associated with content source, and may return the identified audible messages back to control circuitry 304.

For example, the database 600 shown in FIG. 6 may include entries 610 that map each content source identifier field 620 with one or more corresponding audible message fields 630. Each audible message may identify the content source to a user when heard by the user and/or may indicate that the content being received includes an advertisement. The audible messages may be encoded in any format, such as MP3, WAV, etc. The phrase in quotes shown in audible message field 630 represents the phrase that is output by the speakers to be heard by the user. The phrase is referred to as the content of the audible message. In some embodiments, the entries of database 600 may be populated based on information received from media guidance data source 418 (FIG. 4). In some embodiments, the entries of database 600 may be manually entered by the user. In some embodiments, database 600 may be automatically populated locally by control circuitry 304 based on media guidance data associated with each content source.

In some embodiments, when a given content source identifier is associated with multiple audible messages, each may include different content. In some embodiments, one of the audible messages may read back the name or number of the content source (e.g., “Channel 1” or “ESPN”). In some embodiments, one of the audible messages may read back the type of the content source (e.g., “sports channel”). In some embodiments, the audible message may read back the name or number of the content source and the type of the content source (e.g., “ESPN a sports channel”). In some embodiments, the audible message may state that the currently selected content source is currently presenting an advertisement or is in a commercial break. In some embodiments, the audible message may state the name or number of the content source and indicate that the currently selected content source is currently presenting an advertisement or is in a commercial break (e.g., “ESPN is in a commercial break”).

In some embodiments, the audible message may read back a personalized message for the user (e.g., “John this channel is playing a commercial”). The personalized message may be generated and stored by control circuitry 304 in response to a user selecting an option to record a personalized message. For example, the user may instruct control circuitry 304 to record one or more audible messages. After control circuitry 304 records the audible message(s), control circuitry 304 may store the audible message in storage 308. Control circuitry 304 may request input from the user identifying one or more content sources with which to associate the personalized audible message. Control circuitry 304 may store the personalized audible message in an entry in database 600 (FIG. 6).

In some embodiments, control circuitry 304 may select one of the multiple audible messages that are associated with a given content source based on a user profile, user selected setting, or pre-defined default setting. In some implementations, a user profile may be stored in storage 308 indicating a user preference for a type of audible message. In particular, the user profile may indicate that the user has a preference for audible messages that only read out the name or number of the content source. In such circumstances, control circuitry 304 may identify which of the multiple audible messages that are associated with the given content source match the type specified by the user profile. For example, control circuitry 304 may only read out the name or number of the content source. Control circuitry 304 may select that identified audible message as the audible message to output to the user when the user requests to access the content source when that content source is currently receiving content that includes an advertisement.

In some implementations, a user selected setting may be stored in storage 308 indicating a type of audible message. In particular, the user selected setting may indicate that the user has selected audible messages of the type that read out the name or number of the content source along with the type of the content source. In such circumstances, control circuitry 304 may identify which of the multiple audible messages that are associated with the given content source match the type specified by the user. For example, control circuitry 304 may read out the name or number of the content source and the type of the content source. Control circuitry 304 may select that identified audible message as the audible message to output to the user when the user requests to access the content source when that content source is currently receiving content that includes an advertisement.

In some implementations, a default setting may be stored in storage 308 indicating a type of audible message. In particular, the default setting may identify audible messages of the type that read out a statement that indicates that the content currently being received from the selected content source includes an advertisement or is in a commercial break. In such circumstances, control circuitry 304 may identify which of the multiple audible messages that are associated with the given content source match the type specified by the default setting. For example, control circuitry 304 may read out the statement that indicates that the content currently being received from the selected content source includes an advertisement or is in a commercial break. Control circuitry 304 may select that identified audible message type as the audible message to output to the user when the user requests to access the content source when that content source is currently receiving content that includes an advertisement.

At step 570, control circuitry 304 may determine whether an accessibility setting is enabled. In response to determining that the accessibility setting is enabled, the process may proceed to step 580; otherwise, the process proceeds to step 590. In some embodiments, the accessibility setting may be enabled by the user through a user interface. Specifically, a user interface may be presented to the user with accessibility options. One of the options may be a setting that instructs control circuitry 304 to provide an audible message (prompt or notification) that identifies a given content source when the user requests access to the content source. The audible message however is only provided when the requested content source is currently receiving content that includes an advertisement (e.g., is a media asset that is in a commercial break). In response to receiving a user selection enabling this accessibility setting, control circuitry 304 may store an indication in storage 308 of the selection. As such, every time the user requests access to a content source from which an advertisement is currently being received, control circuitry 304 may sound the audible message identifying the selected content source.

In some embodiments, the accessibility setting may be enabled by pressing a dedicated button on a user input device. For example, the user input device may include an accessibility button. The button may be an on-screen selectable option in the case of a user input device being a tablet with a touch sensitive display. In response to receiving a user selection of the accessibility button, control circuitry 304 may automatically enable the accessibility setting. As such, every time the user requests access to a content source from which an advertisement is currently being received, control circuitry 304 may sound the audible message identifying the selected content source.

At step 580, control circuitry 304 may play the retrieved audible message to identify the content source to the user. In some embodiments, the audible message may be played or sounded before any portion of the audio of the content received from the content source is played or sounded. For example, control circuitry 304 may retrieve the audible message identified by database 600 from storage 308. Control circuitry 304 may decode the audible message using a suitable audio decoder and output the audio of the message to the speakers. In some embodiments, control circuitry 304 may retrieve the audible message from a remote server for playback to the user. When multiple audible messages are associated with a given content source, control circuitry 304 may retrieve the audible message selected based on the user profile, user selected setting, or pre-defined default setting for playback or output to a user.

In some embodiments, control circuitry 304 may output the audible message to a second screen device or a portable headset worn by the user. For example, control circuitry 304 may transmit an identifier of the audible message to a second screed device or portable headset worn by the user. When multiple audible messages are associated with a given content source, control circuitry 304 may transmit an identifier of the audible message selected based on the user profile, user selected setting, or pre-defined default setting for playback or output on the second screen device or portable headset. The second screed device or portable headset may retrieve the audible message identified by the identifier from local or remote storage and may sound the message for the user.

At step 590, control circuitry 304 may play the audio portion of the received content. In some embodiments, the content may be a media asset received from the content source using a tuner. The media asset may include an audio component and a video component. When control circuitry 304 is playing the audible message, the audio component may be suppressed, muted and/or replaced with the audible message while the video component is being presented to the user. After the audible message completes being played or output to the user, the audio component may be provided together with the video of the media asset. In some embodiments, the audio component of the media asset may be played or output to the user together or simultaneously with the audible message. For example, the audible message may be played or output to the user using a portable headset (headphones) or second screen device while the audio component of the media asset is output through the speakers of user equipment 300.

It should be understood that the above steps of the flow diagram of FIG. 5 may be executed or performed in any order or sequence not limited to the order and sequence shown and described in the figure. Also, some of the above steps of the flow diagram of FIG. 5 may be executed or performed substantially simultaneously where appropriate or in parallel to reduce latency and processing times.

The above-described embodiments of the present disclosure are presented for purposes of illustration and not of limitation, and the present disclosure is limited only by the claims which follow.

1. A method for assisting a user in content selection, the method comprising:
receiving a user request to access a content source;
receiving content from the content source in response to the user request;
determining whether the content received from the content source is an advertisement;
retrieving an identifier of the content source from memory;
cross-referencing the identifier of the content source with a database of audible messages to retrieve an audible message associated with the content source; and
playing the audible message to identify the content source to the user in response to determining that the content received from the content source is an advertisement.

2. The method of claim 1, wherein the audible message is sounded for the user before an audio portion of the content is output to the user.

3. The method of claim 1 further comprising receiving a user input to enable an accessibility setting, wherein the audible message is played when the accessibility setting is enabled, and wherein playing of the audible message is prevented when the accessibility setting is disabled.

4. The method of claim 1, wherein the content source is a television channel, and wherein the user request is a channel change request.

5. The method of claim 1, wherein determining whether the content received from the content source is the advertisement comprises processing information associated with the received content.

6. The method of claim 5, wherein the information includes closed-caption information associated with the received content.

7. The method of claim 1, wherein the audible message includes a name of the content source and an indication that the content being received is an advertisement.

8. The method of claim 1 further comprising:
sounding the audible message in response to determining that the content received from the content source is an advertisement; and
preventing sounding of the audible message in response to determining that the content received from the content source is not an advertisement.

9. The method of claim 1, wherein a media asset is scheduled for transmission from the content source during a time when the user request to access the content source is received, wherein the media asset includes a commercial segment and a plot segment, wherein the content is a portion of the media asset currently being received from the content source, and wherein the advertisement corresponds to the commercial segment of the media asset.

10. The method of claim 1, wherein the audible message includes a personalized message.

11. A system for assisting a user in content selection, the system comprising:
control circuitry configured to:
receive a user request to access a content source;
receive content from the content source in response to the user request;
determine whether the content received from the content source is an advertisement;
retrieve an identifier of the content source from memory;
cross-reference the identifier of the content source with a database of audible messages to retrieve an audible message associated with the content source; and
play the audible message to identify the content source to the user in response to determining that the content received from the content source is an advertisement.

12. The system of claim 11, wherein the audible message is sounded for the user before an audio portion of the content is output to the user.

13. The system of claim 11 wherein the control circuitry is further configured to receive a user input to enable an accessibility setting, wherein the audible message is played when the accessibility setting is enabled, and wherein playing of the audible message is prevented when the accessibility setting is disabled.

14. The system of claim 11, wherein the content source is a television channel, and wherein the user request is a channel change request.

15. The system of claim 11, wherein the control circuitry is further configured to process information associated with the received content.

16. The system of claim 15, wherein the information includes closed-caption information associated with the received content.

17. The system of claim 11, wherein the audible message includes a name of the content source and an indication that the content being received is an advertisement.

18. The system of claim 11, wherein the control circuitry is further configured to:
sound the audible message in response to determining that the content received from the content source is an advertisement; and
prevent sounding of the audible message in response to determining that the content received from the content source is not an advertisement.

19. The system of claim 11, wherein a media asset is scheduled for transmission from the content source during a time when the user request to access the content source is received, wherein the media asset includes a commercial segment and a plot segment, wherein the content is a portion of the media asset currently being received from the content source, and wherein the advertisement corresponds to the commercial segment of the media asset.

20. The system of claim 11, wherein the audible message includes a personalized message.

21-40. (canceled)
